(19) United States
(12) Patent Application Publication
(10) Pub. No.: US 2008/0045819 A1
(43) Pub. Date: Feb. 21, 2008

(54) Blood-Sugar Level Management System

(75) Inventors: Fumiaki Emoto, Kanagawa (JP); Chiyohiro Hoshikawa, Ehime (JP); Tetsuo Hiraga, Tokushima (JP); Koichi Matsuda, Ehime (JP)

Correspondence Address: MCDERMOTT WILL & EMERY LLP, 600 13TH STREET, NW, WASHINGTON, DC, 20005-3096, US

(73) Assignee: MATSUSHITA ELECTRIC INDUSTRIAL CO., LTD., Kadoma-shi (JP)

(21) Appl. No.: 11/547,145

(22) PCT Filed: Jul. 21, 2005
(86) PCT No.: PCT/JP05/13371

§ 371 Date: Oct. 2, 2006

(30) Foreign Application Priority Data
Jul. 21, 2004 (JP) 2004-212910

(51) Int. Cl. A61B 5/00

(52) U.S. Cl. 600/316; 705/3

(57) ABSTRACT

There is provided a mobile blood-sugar level measuring system enabling a diabetic patient to accurately and easily perform management of blood-sugar level measurement timing, management of changes in blood-sugar level, and further prediction of a future blood-sugar level. The blood-sugar level measuring system is formed by using a blood-sugar level processing device including a data-arithmetic-processing unit for arithmetically processing the measurement-data, an external data importing unit for entering and accumulating external data on blood-sugar level, a display device for displaying the result of the arithmetic-processing, and a display device control unit for controlling the display device, in combination with a conventional, mobile blood-sugar level monitoring device.

### TECHNICAL FIELD

The present invention relates to blood-sugar level measuring systems, in which a blood-sugar level of a diabetic patient can be measured, and the measurement data of the blood-sugar level can be managed for an easy usage by diabetic patients and medical doctors.

### BACKGROUND ART

Recently, the number of diabetic patients is rapidly increasing. Diabetes is a metabolic disorder characterized by hyperglycemia caused by insulin secretory dysfunction or underfunction, and is roughly grouped under type 1 (insulin-dependent, some are slowly progressive diabetes starting from non-insulin-dependent) diabetes and type 2 (non-insulin-dependent) diabetes. Further, type 2 diabetes is categorized into those characterized by obesity with hyperinsulinism and insulin resistance (insulin functional disorder), characterized by non-obesity with insulin secretion disorder, and those with both disorders.

The treatment method for diabetes includes exercise-approach, diet-approach, and pharmaceutical approach, and in conducting any approach, keeping track of patient's blood-sugar level measurement values such as blood pressure and blood-sugar level; energy amount consumed in exercises; meal intake amount; and the like is necessary. Particularly, accuracy is required for conducting management of meal intake timing and intake amount, management of blood-sugar level measurement timing, management of changes in blood-sugar level, prediction of a future blood-sugar level, and a grasp on the energy amount by nutrient to be taken.

Thus, in exercise-approach, patients have been carrying out a work of consuming energy as prescribed by a medical institute by running, swimming, or an exercise using an exercise assisting device such as treadmills, and recording in handwriting or so the consumed energy amount. Also, in meal approach, patients themselves have been carrying out a work of estimating the food material and its weight visually, calculating the energy amount by nutrient based on the Tables of Food Composition, and recording by handwriting or so. Additionally, in realities, the measurement-timing for the blood-sugar level has been checked by using such records and ordinary clocks.

For such problems, in patent publication document 1, for example, a mobile terminal for self-health-management and a support system for self-health-management have been proposed, for supporting the exercise-approach and the meal-approach effectively and for reducing a burden on patients. Also, a health management system is disclosed in patent publication document 2, in which data on blood-sugar level is sent to a computer of a hospital by a mobile phone, and a corresponding prescription is automatically sent to the patient's mobile phone based on the measured blood-sugar level data sent.

However, the self-management support system in patent publication document 1 is, considered as large-sized and unsuitable for carrying along, mainly aimed for energy amount management by nutrient contained in food material, and without particular functions for patients to recognize easily the results from measuring and managing the blood-sugar level.

The health management system of patent publication document 2 is as well considered large-sized and unsuitable for carrying along, and prescriptions are just sent from computers to mobile phones of patients: measurement and management of blood-sugar level, and its results are unavailable for patients to conveniently and easily use for the exercise-approach and the meal-approach.

Additionally, although as a conventional blood-sugar monitoring device, the one including a monitoring unit, a data-accumulation unit, a time-management unit, and a display unit for displaying necessary information (numeral value, time, and the like) is known, its functions are just enough for displaying the blood-sugar level after completing the measurement and the blood-sugar level measured in the past along with the date and time of the measurement, and the display contents and display method are not effectively applicable to the maximum for patient's meal-approach and exercise-approach.

Patent Publication Document 1:

Japanese Laid-open Patent Publication No. 2002-222263

Patent Publication Document 2:

Japanese Laid-Open Patent Publication No. 2003-180637

### DISCLOSURE OF INVENTION

### The Problem to be Solved by the Invention

Thus, an object of the present invention is to provide a simple blood-sugar level measuring system, in which management of blood-sugar level measurement timing, management of changes in blood-sugar level, prediction of a future blood-sugar level, and further, management of meal intake timing and intake amount, can be carried out accurately and easily, for assisting the meal-approach and the exercise-approach to be effective for diabetic patients, regardless of whether the patient is type 1 or type 2.

### Means for Solving the Problem

To solve the problems as noted in the above, the present invention achieves a more easy-to-use blood-sugar level measuring system for diabetic patients by using a portable blood-sugar meter having conventional structure and an external blood-sugar level processing device in combination. That is, the blood-sugar level measuring system according to the present invention comprises a combination of a blood-sugar level monitoring device and a blood-sugar level processing device; the devices are connectable via a first interface unit of the former and a second interface unit of the latter; and at least one of the measurement-data, the result of processing, and the result of arithmetic-processing can be transmitted to and received from the devices and be displayed.

The blood-sugar level monitoring device comprises:

a blood-sugar monitoring unit for measuring a blood-sugar level of an examinee;

a measurement-data accumulation unit for accumulating the measured blood-sugar level as measurement-data;

a measurement-control data-pre-processing unit for pre-processing the measurement-data;

a first display device for displaying the pre-Processing result;

a first display-device control-unit for controlling the first display device;

a time-management unit for managing measurement timing of the blood-sugar level;

a control switch for controlling the monitoring unit, the measurement-data accumulation unit, the measurement-data pre-processing unit, the first display device, the first display-device control-unit, and the time-management unit; and

a first interface unit.

On the other hand, the blood-sugar level processing device comprises:

a data-arithmetic-processing unit for arithmetically processing the measurement-data from the measurement-control data-pre-processing unit or external data;

a second display device for displaying the result of the arithmetic processing;

a second display-device control-unit for controlling the second display device; and

a second interface unit.

Thus, the present invention also relates to a blood-sugar level monitoring device and a blood-sugar level processing device used for the above blood-sugar level measuring system.

The data-arithmetic-processing unit preferably is provided with a means for creating a graph in which the measured blood-sugar level is plotted against time based on the measurement-data; and the second display device is preferably provided with a means for displaying the graph.

Additionally, the data-arithmetic-processing unit preferably is provided with a means for creating a graph in which the measured blood-sugar level is plotted against elapsed time from immediately after a start of a breakfast, lunch, or dinner intake based on the measurement-data; and the second display device preferably is provided with a means for displaying the graph.

The data-arithmetic-processing unit preferably is provided with a period-changing means for changing a period of the time by the control switch.

The measurement-data accumulation unit is preferably provided with a means for changing the accumulation period of the measurement-data.

The second display device is preferably provided with a means for plotting the target blood-sugar level on the graph in cooperation with the data-arithmetic-processing unit.

Additionally, the data-arithmetic-processing unit preferably is provided with a means for creating a smoothed blood-sugar spline curve (an approximated spline curve in which Changes in the blood-sugar level over time are smoothed: approximated spline curve of a blood glucose response) in which the measured blood-sugar level is plotted against time based on the measurement-data; and the second display device is preferably provided with a means for displaying the smoothed blood-sugar spline curve on the graph.

The data-arithmetic-processing unit preferably is provided with a means for creating a target blood-sugar response curve (a curve of changes in the target blood-sugar level over time: care target of a blood glucose response) showing a target blood-sugar level against time by assigning the measurement-data in a higher order approximation function; and the second display device is preferably provided with a means for displaying the target blood-sugar response curve.

The measurement-data to be assigned preferably comprises three measured blood-sugar levels including at least a blood-sugar level at a meal-start and a blood-sugar level after an elapse of two hours from the meal-start.

Additionally, the measurement-data to be assigned preferably comprises a blood-sugar level at a meal-start, a peak blood-sugar level, a time period from the meal-start to the point when the peak blood-sugar level is obtained, and a blood-sugar level after an elapse of two hours from the meal-start.

The measurement-data to be assigned may also comprise a blood-sugar level at a meal-start, a blood-sugar level after an elapse of one hour from the meal-start, and a blood-sugar level after an elapse of two hours from the meal-start.

The data-arithmetic-processing unit preferably is provided with a means for creating a target blood-sugar response curve showing target blood-sugar levels against time by assigning the measurement-data accumulated in a certain period in the past in a higher order approximation function; and the second display device preferably is provided with a means for displaying the target blood-sugar response curve.

The external data includes a target blood-sugar level set by a medical doctor.

The data-arithmetic-processing unit preferably is provided with a means for creating a target blood-sugar response curve showing target blood-sugar levels against time by assigning the external data in a higher order approximation function; and the second display device preferably is provided with a means for displaying the target blood-sugar response curve.

The data-arithmetic-processing unit preferably is provided with a means for calculating a predicted blood-sugar level after an elapse of a certain time based on the measurement-data accumulated in a certain period in the past or a latest measurement-data; and the second display device is preferably provided with a means for displaying the predicted blood-sugar level.

The control switch preferably includes a timing input switch for inputting the measurement timing; and the first display device is preferably provided with a means for displaying the measurement timing or a time till the measurement timing in cooperation with the time-management unit.

The first display device and/or the second display device is preferably provided with a means for displaying a latest of the measured blood-sugar levels by blinking, or as a predetermined mark and are preferably provided with a means for scrolling the displayed contents to the horizontal direction or to the vertical direction.

Further, the first display device and/or the second display device is preferably provided with a means for rotating the displayed contents upside-down.

### EFFECT OF THE INVENTION

The blood-sugar level measuring system according to the present invention is formed by a combination of a blood-sugar level monitoring device and a blood-sugar level processing device. Conventional blood-sugar level management can be carried out by the blood-sugar level monitoring device, and further, by connecting the monitoring device with an external blood-sugar level processing device, management of blood-sugar level measurement timing, management of changes in blood-sugar level, prediction of a future blood-sugar level, and further management of meal intake timing and intake amount can be carried out accurately and joyfully. Thus, the blood-sugar level measuring system is very effective in assisting diabetic patients' meal-approach and exercise-approach.

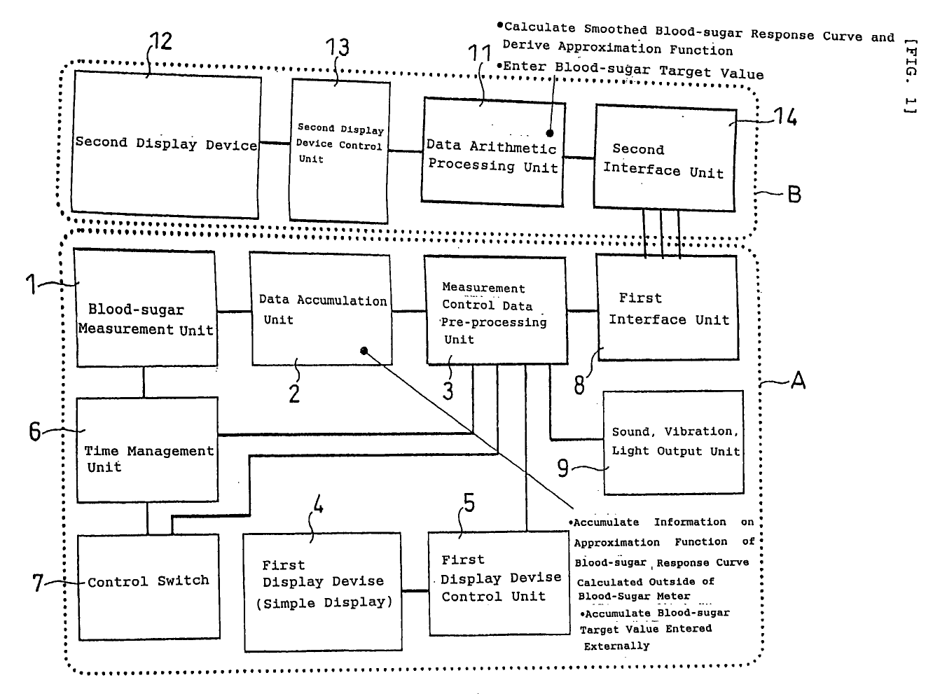

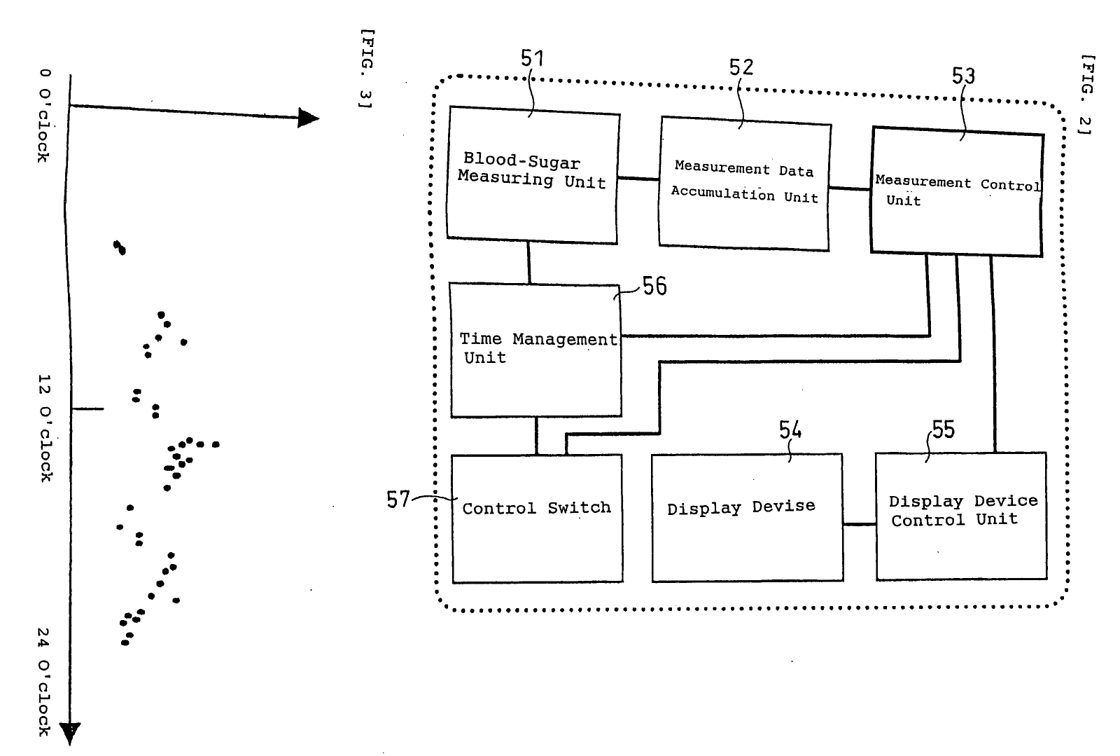

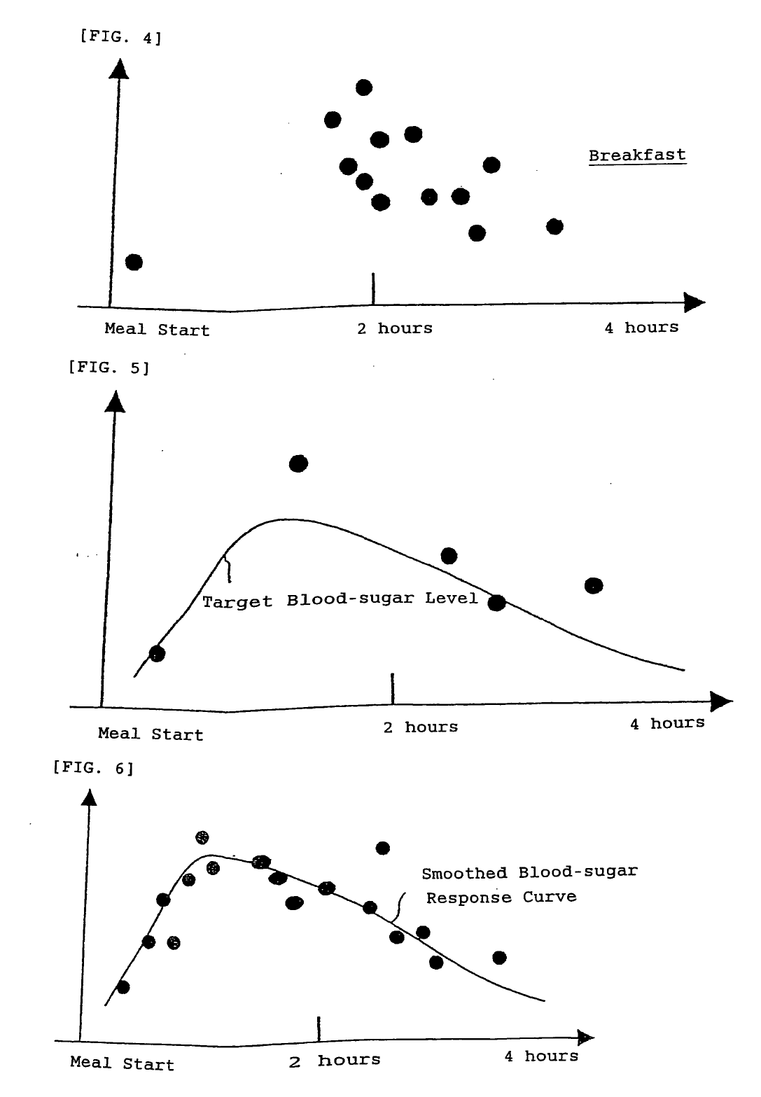

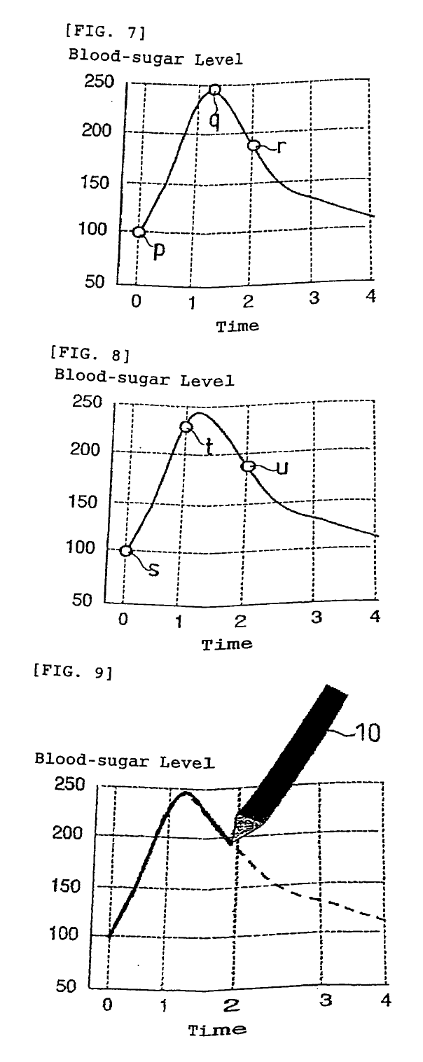

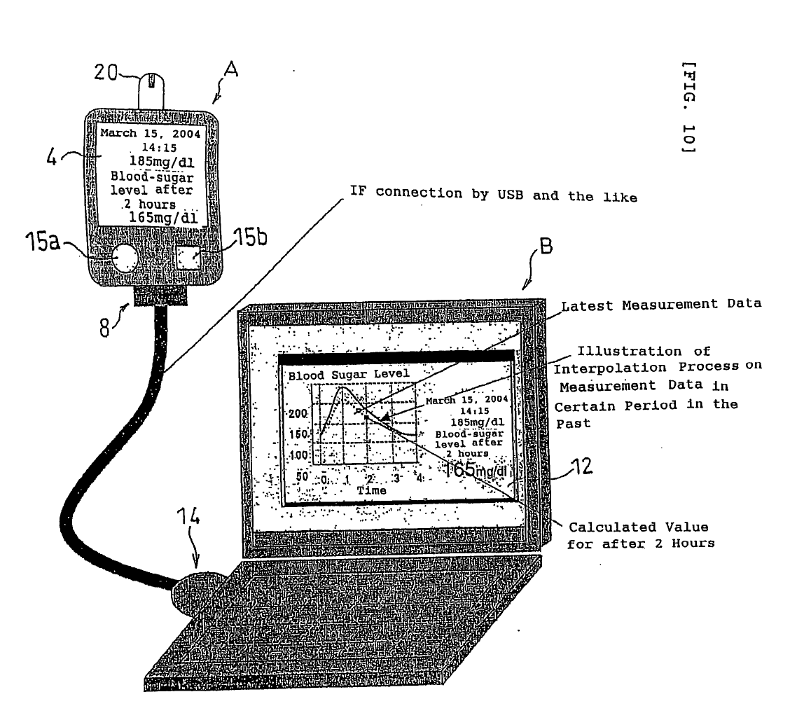

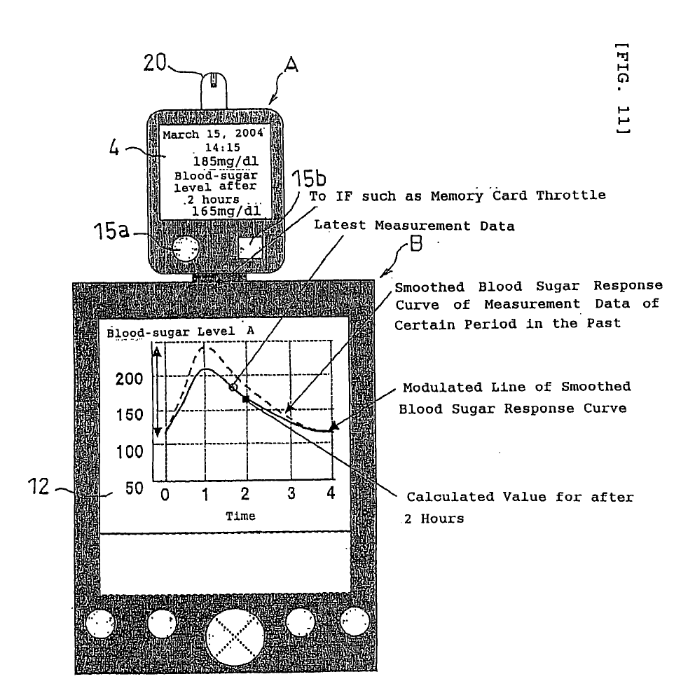

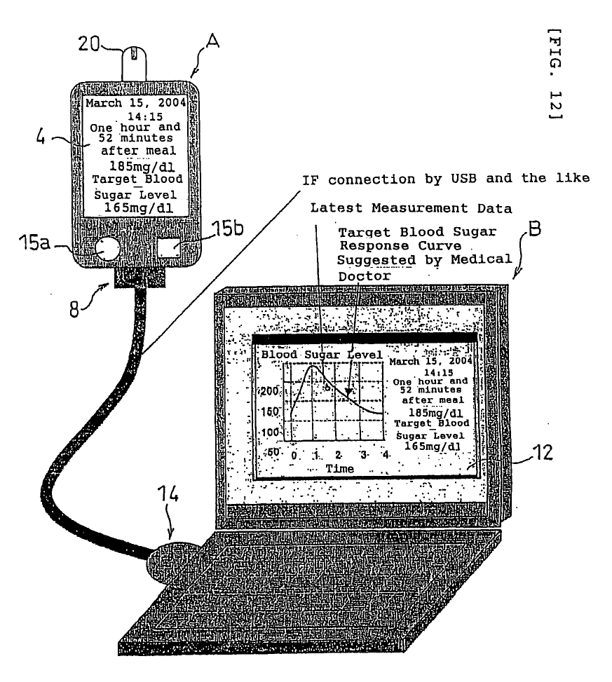

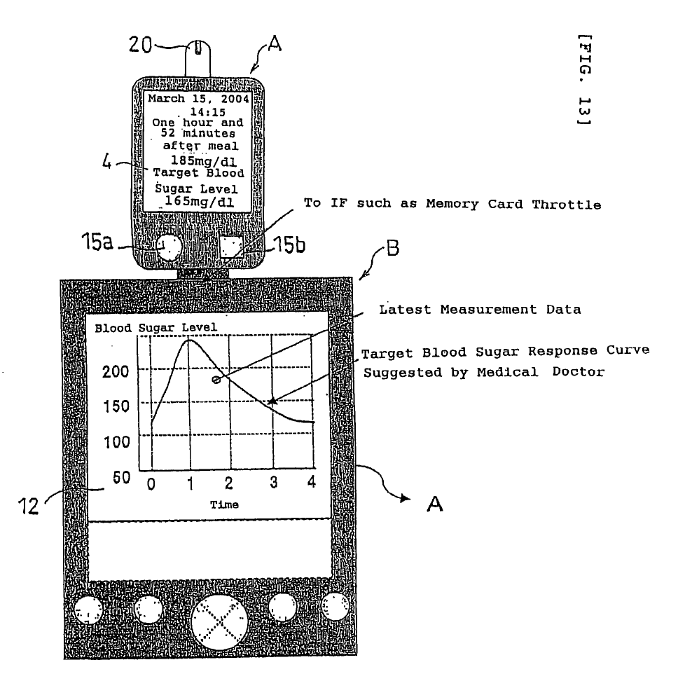

### BRIEF DESCRIPTION OF THE DRAWINGS

[FIG. 1] A block diagram illustrating a structure of a blood-sugar level measuring system according to the present invention.

[FIG. 2] A block diagram illustrating a structure of a conventional blood-sugar level monitoring device.

[FIG. 3] An example of a graph made by a data-arithmetic-processing unit 11 based on measurement data.

[FIG. 4] Another example of a graph made by a data-arithmetic-processing unit 11 based on measurement data.

[FIG. 5] Another example of a graph made by a data-arithmetic-processing unit 11 based on measurement data.

[FIG. 6] Still another example of a graph made by a data-arithmetic-processing unit 11 based on measurement data.

[FIG. 7] A graph including a target blood-sugar response curve obtained by a predetermined higher order approximation functions showing time and blood-sugar levels.

[FIG. 8] A graph including another target blood-sugar response curve obtained by a predetermined higher order approximation function, showing time and blood-sugar levels.

[FIG. 9] A diagram illustrating a manner in which a target blood-sugar response curve is inputted at a display device of an external data-input device with a pen 11.

[FIG. 10] A schematic diagram illustrating an embodiment of a blood-sugar level management system in accordance with the present invention.

[FIG. 11] A diagram illustrating another embodiment of a blood-sugar level management system in accordance with the present invention.

[FIG. 12] A schematic diagram illustrating still another embodiment of a blood-sugar level management system in accordance with the present invention.

[FIG. 13] A schematic diagram illustrating still another embodiment of a blood-sugar level management system in accordance with the present invention.

### BEST MODE FOR CARRYING OUT THE INVENTION

A structure of a blood-sugar level measuring system in accordance with the present invention is explained. FIG. 1 is a schematic diagram of a blood-sugar level measuring system according to the present invention. The blood-sugar level measuring system according to the present invention is formed with a blood-sugar level monitoring device A and a blood-sugar level processing device B.

The blood-sugar level monitoring device A has small and portable shape and size, and comprises: a blood-sugar monitoring unit 1 to which a biosensor (not shown) is inserted for measuring a blood-sugar level; a measurement-data accumulation unit 2 for accumulating measurement data; a time-management unit 6 for managing measurement protocol's time-management and measurement time; and a control switch 7 for controlling the monitoring unit 1, the measurement-data accumulation unit 2, and the time-management unit 6. A measurement-control data-pre-processing unit 3 controls the blood-sugar monitoring unit 1, the measurement-data accumulation unit 2, the time-management unit 6, and the control switch 7. Necessary information such as a measurement-result and time is shown at a first display device 4 while being controlled by a first display-device control-unit 5.

By inserting a biosensor into the monitoring unit 1, and by starting a metering function of the blood-sugar monitoring unit 1, when a blood is put on the biosensor, a voltage application and a current application are carried out based on a predetermined measurement protocol to measure the blood-sugar level. The measured blood-sugar level is stored in the measurement-data accumulation unit 2 as measurement-data along with the measurement date. After the measurement, the blood-sugar level is shown as text at the first display device 4 with a control from the first display-device control-unit 5. By using the control switch 7, a past blood-sugar level can also be shown along with its date and time.

However, such conventional function merely enables patients and medical doctors to check individual blood-sugar level and its date and time as textual information at the first display device 4, and statistics on blood-sugar levels in a certain time period in the past and a target blood-sugar level, for example, cannot be checked. Thus, the blood-sugar level measuring system according to the present invention is formed by a combination of the blood-sugar level monitoring device A having functions same as conventional ones, and the blood-sugar level processing device B to be used while being connected with the device A; and a smoothed blood-sugar response curve based on the measured blood-sugar level and a target blood-sugar response curve based on a target blood-sugar level can be shown.

The blood-sugar level processing device B may be for example a personal digital assistant (PDA), a mobile personal computer, or a mobile phone, and has a second display device 12, a second display-device control-unit 13, a data-arithmetic-processing unit 11 capable of importing external data on blood-sugar level, and a second interface unit 14 to be connected with a first interface unit 8 of the blood-sugar level monitoring device A. Conducting various arithmetic processing for blood-sugar levels to obtain statistical data with the blood-sugar level processing device B, and then showing the results with the blood-sugar level monitoring device A, the system can be useful for the examinee's exercise-approach and meal-approach.

FIG. 2 shows a structure of a conventional blood-sugar level monitoring device. The conventional blood-sugar level monitoring device has a blood-sugar monitoring unit 51 in which a biosensor (not shown) is inserted for measuring a blood-sugar level, a measurement-data accumulation unit 52 for accumulating the measurement-data, and a time-management unit 56 for managing measurement protocol's time-control and measurement time, a control switch 57 for controlling the blood-sugar monitoring unit 51, the measurement-data accumulation unit 52, and the time-management unit 56. A measurement control unit 53 controls the blood-sugar monitoring unit 51, the measurement-data accumulation unit 52, the time-management unit 56, and the control switch 57. Necessary information such as measurement-results and time is shown in a display device 54, with a control by a display device control unit 55. However, shown in the display device 54 with a control by the display device control unit 55 are blood-sugar levels and the measurement dates as textual information.

On the other hand, as shown in FIG. 1, the blood-sugar level measuring system according to the present invention comprises the blood-sugar level monitoring device A and the blood-sugar level processing device B, which are connectable via a first interface unit 8 and a second interface unit 14, and as described in the above, various information can be provided to examinees by using the blood-sugar level measurement-data and external data. The blood-sugar level measuring system according to the present invention is described further in detail below, by using FIG. 1.

(1) Blood-Sugar Level Monitoring Device

The blood-sugar monitoring unit 1 is a part for measuring a blood-sugar level of an examinee (patient) by using a biosensor and the like. For the biosensor, the one conventionally used for measuring a blood-sugar level may be used. For example, a chip biosensor described in Japanese Laid-Open Patent Publication No. Hei 2-062952 is preferably used.

The biosensor described in the above Publication is made by forming an electrode system comprising a working electrode, a counter electrode, and a reference electrode on an insulating base plate by a method of screen printing or the like; and forming an enzyme reaction layer contacting the electrode system and including a hydrophilic polymer, an oxidoreductase (glucose oxidase), and an electron acceptor.

When a sample solution (blood) including a substrate (glucose) is dropped onto the enzyme reaction layer of the biosensor thus made, the enzyme reaction layer is dissolved and the glucose oxidase and substrate are reacted, thereby reducing the electron acceptor. After the completion of the enzyme reaction, the reduced electron acceptor is Oxidized electrochemically, and the concentration of the substrate (blood-sugar level) in the sample solution can be determined from a value of oxidation current obtained at this time.

Then, the measurement-data accumulation unit 2 accumulates the measured blood-sugar level (measurement data) obtained by the blood-sugar monitoring unit 1, a smoothed blood-sugar response curve, a target blood-sugar level, and the like calculated in the blood-sugar level processing device B. Thus, for the measurement-data accumulation unit 2, a storage medium capable of reading/writing such as a semiconductor memory and the like may be used.

For the first display device 4 in the blood-sugar level monitoring device A, as in conventional case, the one that can show current or past blood-sugar level along with date and time as textual information will suffice, and is controlled by a first display-device control-unit 5. An ordinary liquid crystal display device may be used for the first display device 4.

Additionally, controlling the first display device 4 by the first display-device control-unit 5 can be effective for exhibiting functions of selecting a particular portion such as the latest measured blood-sugar level to be blinked or displayed with a predetermined mark, scrolling the displayed content such as a graph displayed in the display device 4 in the horizontal direction (or the vertical direction), and rotating (inversing) 180 degrees. For the first display-device control-unit 5, for example, an image signal processing IC may be used.

The time-management unit 6 for managing the timing of a blood-sugar level measurement by the blood-sugar monitoring unit 1 plays a role to calculate and manage the measurement timing in cooperation with the measurement-control data-pre-processing unit 3, and to notify the patient the measurement timing by alarming in cooperation with the first display device 4. For the time-management unit 6, for example, a clock including a quarts oscillator may be used.

The control switch 7 is for controlling the monitoring unit 1, the measurement-data accumulation unit 2, the measurement-control data-pre-processing unit 3, the first display device 4, the first display-device control-unit 5, and the time-management unit 6.

For example, via the control switch, patients and medical doctors measure a blood-sugar level with the blood-sugar monitoring unit 1, select the measurement data (for example, the measurement data of a certain period in the past) to be accumulated in the measurement-data accumulation unit 2, and select the measurement-data to be sent from the measurement-control data-pre-processing unit 3 to the blood-sugar level processing device B for processing. Additionally, information on target blood-sugar levels and smoothed blood-sugar response curves received from the blood-sugar level processing device B is selected and shown in the first display device.

The control switch 7 is preferably provided with a timing-input switch for entering a measurement timing. This is used for diabetic patients to enter the time of the meal-start in the blood-sugar level monitoring device. The blood-sugar level can be measured immediately before meal, and the time can be entered as meal-start time.

Further, an output unit 9 for generating at least one selected from the group consisting of sound, vibration, and light is preferably connected to the first display-device control-unit 5, and the first display-device control-unit 5 preferably is provided with a means for controlling the Output unit. That is, a latest of the measured blood-sugar level and a predicted blood-sugar level may be outputted by a sound. According to this, a measurement-timing for a blood-sugar level after meal can be notified to the patient. Such measurement timing is not only limited to the time for the blood-sugar level measurement after meal, for example, and can be set to 5 minutes before the time of the measurement.

Also, at least one of sound, vibration, and light can be generated by every determined time period for example by every 2 hours, by the management of the time-management unit 6.

The first interface unit 8 is formed with a connecting terminal unit and an interface circuit unit, for exchanging data with external units as well as for connecting the blood-sugar level monitoring device A with the blood-sugar level processing device B. The connection may be enabled for example by USBs.

(2) Blood-sugar Level Processing Device

The blood-sugar level processing device B according to the present invention is formed with a second display device 12, a second display-device control-unit 13, a data-arithmetic-processing unit 11, and a second interface unit 14. For example, PDA (Personal Digital Assistant), PC (Personal Computer), and mobile phone may be mentioned.

The data-arithmetic-processing unit 11 in the blood-sugar level processing device B plays the most characteristic role in the blood-sugar measuring system of the present invention, and its arithmetic-processing method is particularly characteristic.

The data-arithmetic-processing unit 11 is firstly capable of arithmetically processing the measurement-data from the blood-sugar level monitoring device A and/or external data, and creating (mapping) a graph from the results of the arithmetic-processing. Secondly, from the measurement-data and the external data, a future target blood-sugar level, a predicted blood-sugar level, and a measurement timing can be calculated as well.

Functions of the data-arithmetic-processing unit 11 are described in detail below. For the data-arithmetic-Processing unit 11, may be used is a microprocessor or the like including a CPU and a memory, for memorizing and executing programs and applications realizing various functions described in the specification of the present invention.

The second display device 12 in the blood-sugar level processing device B not only shows current and past blood-sugar levels of current or past along with its date and time as textual information as in the conventional ones, but also shows a graph to be mentioned later, and is controlled by the second display-device control-unit 13.

Additionally, controlling the second display device 12 by the second display-device control-unit 13 can be effective for exhibiting functions of selecting a particular portion such as a latest of the measured blood-sugar level to be blinked or displayed with a predetermined mark, scrolling the displayed content such as a graph displayed in the display device 4 in the horizontal direction (or the vertical direction), and rotating (inversing) 180 degrees. For the second display-device Control-unit 13, for example, an image signal processing IC may be used, as for the first display-device control-unit.

The second display device 12 shows the result of the arithmetic processing by the data-arithmetic-processing unit 11, a graph showing the result, the external data including a target blood-sugar level, average values of the external data and measurement-data, a target blood-sugar response curve obtained from external data, a smoothed blood-sugar response curve obtained from measurement-data, a predicted blood-sugar level, and measurement timing. Based on this, patients and further medical doctors can compare data with external data, grasp the measurement-data of blood-sugar levels from various viewpoints, and make use of it for meal-approach and exercise-approach for diabetes. An ordinary liquid crystal display device may be used for the second display device 12.

The second interface unit 14 may be formed with a connecting terminal unit and an interface circuit unit, as the first interface unit 8.

To the data-arithmetic-processing unit 11, external data set by medical doctors can be entered, and the external data can be stored.

The external data include, blood-sugar measurement data (calorie vs. blood-sugar level) by regular meal, blood-sugar measurement data (calorie vs. blood-sugar level) by commercially available diabetic meal, a target blood-sugar level set by a medical doctor for a patient, accumulation of the past measurement data of the patient, consumed calories, meal calories, the PFC ratio (ratio between protein, fat, and carbohydrate), meal menu, the heart rate appropriate for the exercise-approach, an exercise amount (number of steps), and other various data needed by diabetic patients.

Basic functions of the data-arithmetic-processing unit 11 are briefly explained.

First, the data-arithmetic-processing unit 11 preferably has a means or function for creating a graph in which the measured blood-sugar level is plotted against time based on the measurement data sent from the blood-sugar level monitoring device A, and is able to display the graph on the second display device 12.

FIG. 3 shows an example of the graph thus created. FIG. 3 is a graph obtained by plotting time on the horizontal axis, and plotting the measured blood-sugar level on the vertical axis based on the measurement data.

Also, the data-arithmetic-processing unit 11 preferably is provided with a means or function for creating a graph in which the measured blood-sugar level is plotted against elapsed time from immediately after a start of a breakfast, lunch, or dinner intake (meal-start time) based on the measurement data, and is able to display the graph on the second display device 12.

FIG. 4 shows an example of the graph thus created. FIG. 4 is a graph obtained by plotting the measured blood-sugar level on the vertical axis and elapsed time from immediately after the breakfast intake (meal-start time) on the horizontal axis based on the measurement data.

Also, the data-arithmetic-processing unit 11 preferably has a means or function for plotting a target blood-sugar response curve on the graph, the curve illustrating a target blood-sugar level obtained from external data imported externally such as for example a target blood-sugar level set by a medical doctor for the patient. Based on such graph, the patient can check if his/her own current blood-sugar level is good or not immediately.

FIG. 5 shows an example of the graph thus created. In FIG. 5, time elapsed from immediately after the breakfast intake (meal-start time) is plotted on the horizontal axis, and the measured blood-sugar level is plotted on the vertical axis based on the measurement data, and further, a target blood-sugar response curve showing a target blood-sugar level set by for example medical doctors or the like is shown. Based on such graph, the patient can check if his blood-sugar level is good or not against the elapsed time after the meal-start.

Further, the data-arithmetic-processing unit 11 preferably has a means or function for creating a smoothed blood-sugar response curve showing tendencies for the measured blood-sugar level against time by obtaining a higher order approximation function based on the measurement data with a smoothing process using the least squares method or the Gaussian Kernel method, or a smoothing process using running median, and as shown in FIG. 6, the second display device 12 preferably shows the curve on the graph.

For the higher order approximation function, for example, aX⁴+bX³+cX²+dX+e may be mentioned. The period and number of the measurement data used for the Smoothing and interpolation processes can be set arbitrary.

A creation of a target blood-sugar response curve, an important new function of the data-arithmetic-processing unit 11, is explained next.

The data-arithmetic-processing unit 11 preferably has a means or function for creating a target blood-sugar response curve made up by target blood-sugar levels against time, based on the measurement data accumulated in the measurement-data accumulation unit 2 of the blood-sugar level monitoring device A and the measurement data imported as external data, by for example a linear interpolation method, the Spline interpolation method, or the Lagrange interpolation method, and the second display device 12 preferably displays the target blood-sugar response curve.

For the higher order approximation function, for example, aX⁴+bX³+cX²+dX+e may be mentioned.

The measurement data used for creating a target blood-sugar response curve is explained here. The curves shown in FIGS. 7 and 8 are a target blood-sugar response curve obtained by the Spline interpolation method, showing time and blood-sugar levels.

First, as shown in FIG. 7, the measurement data to be assigned preferably include three measured blood-sugar levels including at least a blood-sugar level p at a meal-start and a blood-sugar level r at 2 hours after the meal-start. More particularly, in addition to the blood-sugar levels p and r, a peak blood-sugar level q, and time taken for obtaining the peak blood-sugar level q are preferably included.

This is because the peak blood-sugar level is important for diagnosis of diabetes, not only to patients but also from the viewpoint of medical doctors.

Also, as shown in FIG. 8, the measurement data to be used may include a blood-sugar level s at a meal-start, a blood-sugar level t at 1 hour after the meal-start, and a blood-sugar level u at 2 hours after the meal-start.

In this case, the peak blood-sugar level is not necessarily measured, and it is preferable in the sense that a burden on patients with regard to blood-sugar level measurement can be lessened.

Also, as shown in FIG. 9, the target blood-sugar response curve may be entered, when a touch panel display device is used as the second display device 12 of the blood-sugar level processing device B, with a pen 10 (or a mouse) arbitrary, and the data can be sent to the blood-sugar level monitoring device and displayed. In such case, for example, a target blood-sugar response curve can be created on the spot while a medical doctor is giving an explanation to the patient face to face upon patient's visit to a medical institute.

In the following, embodiments of the blood-sugar level management system according to the present invention are described with reference to the drawings. Explained in here are functions of the blood-sugar level monitoring device A and the contents shown in the first display device 4 based on such functions, as well as functions of the data-arithmetic-processing unit 11 in the blood-sugar level processing device B and the contents shown in the second display device 12 based on such functions.

### EMBODIMENT 1

FIG. 10 is a schematic diagram illustrating an embodiment of a blood-sugar level management system according to the present invention. A biosensor 20 is inserted into a blood-sugar level monitoring device A of the blood-sugar level management system: a second display device 12 of a blood-sugar level processing device B shows a point indicating the measured blood-sugar level (a latest measurement-data) and a target blood-sugar response curve obtained by the Spline interpolation method with a data-arithmetic-processing unit 11, based on for example measurement-data of a certain period in the past accumulated in a data accumulation unit 2. Also, with control switches 15a and 15b, the blood-sugar level monitoring device A can be controlled, a kind and a period of the measurement data to be used for the arithmetic process can be selected, and the measurement data can be entered manually.

The target blood-sugar response curve can be created by various methods as mentioned above. By showing both the target blood-sugar response curve and the latest measurement data, data can be provided for use by patients and medical doctors.

In FIG. 10, the horizontal axis indicates time, setting a meal-start as the starting point. Such display can be shown by breakfast, lunch, and dinner.

The first display device 4 and the second display device 12 may show, other than the measurement date and the latest measurement-data, a predicted blood-sugar level after two hours that can be predicted from the target blood-sugar response curve.

In FIG. 10, the blood-sugar level monitoring device A and the blood-sugar level processing device B are connected via the first interface unit 8 and the second interface unit 14: the blood-sugar level monitoring device A by itself achieves functions similar to that of conventional blood-sugar meter, and may be provided with functions for calculating a predicted measurement value of two hours later, and for counting down till the measurement time, for example.

Additionally, since displaying the measurement data by the period of 24 hours reduces viewability, the second display device 12 may be provided with a function of scrolling in the horizontal axis (arrow) direction so that the measurement data of specific 2 to 6 hours can be displayed.

### EMBODIMENT 2

FIG. 11 is a diagram illustrating another embodiment of a blood-sugar level management system in accordance with the present invention. A biosensor 20 is inserted into a blood-sugar level monitoring device A: a second display device 12 shows a point showing a latest measured blood-sugar level, and a smoothed blood-sugar response curve obtained by the leas-t squares method with a data-arithmetic-processing unit 11, based on the measurement-data accumulated in a certain period in the past.

Further, a predicted blood-sugar level after an elapse of two hours is calculated from the smoothed blood-sugar response curve and the measured blood-sugar level, to show the predicted blood-sugar level. Such an embodiment has merits in that patients can achieve visual comprehension in his target management.

The predicted blood-sugar level after two hours can be obtained by a calculation based on a slope in the proximity of a predetermined time in the smoothed blood-sugar response curve. Additionally, as shown in FIG. 11, a predicted blood-sugar level after an elapse of two hours is calculated by obtaining a modulated line by condensing or extending a difference of the maximum value and the minimum value of the smoothed response curve (arrow A) (condensing in FIG. 11), so that the latest measurement-data takes a position on the modulated line.

The predicted blood-sugar level here is a blood-sugar level predicted for a predetermined time: for example, a blood-sugar level after two hours and a blood-sugar level with an empty stomach before a breakfast can be calculated as 150 mg/dl and 1100 mg/dl, respectively.

In this case as well, since displaying the measurement data by the period of 24 hours reduces viewability, the display device 4 may be provided with a function of scrolling in the horizontal axis (arrow) direction so that the measurement data of specific 2 to 6 hours can be displayed. Also, with control switches 15a and 15b, the blood-sugar level monitoring device A can be controlled, a kind and a period of the measurement data to be used for the arithmetic process can be selected, and the measurement data can be entered manually. The smoothed blood-sugar response curve may be created by various methods as described above, and for the latest measurement data and a predicted blood-sugar level, blinking, a changed mark, and the like can be used as well for easier recognition.

### EMBODIMENT 3

FIG. 12 is a schematic diagram illustrating another embodiment of a blood-sugar level management system in accordance with the present invention.

A biosensor 20 is inserted into a blood-sugar level monitoring device A of the blood-sugar level management system: a second display device 12 of a blood-sugar level processing device B shows a point indicating a measured blood-sugar level (latest measurement-data), and a target blood-sugar response curve relating to a target blood-sugar level suggested by a medical doctor, for example.

With control switches 15a and 15b, the blood-sugar level monitoring device A can be controlled, a kind and a period of the measurement data to be used for the arithmetic process can be selected, and the measurement data can be entered manually.

The target blood-sugar response curve can be created by the various methods described in the above. By showing both the target blood-sugar response curve and the latest measurement data, data can be provided for use by patients and medical doctors.

In FIG. 12, the horizontal axis indicates time, setting a meal-start as the starting point. Such display can be shown by breakfast, lunch, and dinner.

The first display device 4 and the second display device 12 may show, other than the measurement date and the latest measurement-data, a target blood-sugar level at a predetermined time after a meal (after one hour and 52 minutes later in FIG. 12) by a calculation based on the target blood-sugar response curve. The calculation method can be the same as the case with a predicted blood-sugar level after two hours in FIG. 11. That is, even when the patient was unable to conduct the measurement at time X suggested by the medical doctor and the measurement time Shifted by several minutes to several ten minutes, a target blood-sugar level at a predetermined time X can be calculated based on the target blood-sugar response curve and displayed.

In FIG. 12, the blood-sugar level monitoring device A and the blood-sugar level processing device B are connected via the first interface unit 8 and the second interface unit 14: the blood-sugar level monitoring device A by itself achieves functions similar to that of conventional blood-sugar meter, and may be provided with functions for calculating a predicted measurement value of two hours later, and for counting down till the measurement time, for example.

Additionally, since displaying the measurement data by the period of 24 hours reduces viewability, the second display device 12 may be provided with a function of Scrolling in the horizontal axis (arrow) direction so that the measurement data of specific 2 to 6 hours can be displayed.

### EMBODIMENT 4

FIG. 13 is a schematic diagram illustrating still another embodiment of a blood-sugar level management system in accordance with the present invention.

A biosensor 20 is inserted into a blood-sugar monitoring device A of a blood-sugar level management system: a second display device 12 of a blood-sugar level processing device B shows a point showing a measured blood-sugar level (latest measurement-data), and a target blood-sugar response curve relating to a target blood-sugar level suggested by a medical doctor, for example.

Additionally, with control switches 15a and 15b, the blood-sugar level monitoring device A can be controlled, a kind and a period of the measurement data to be used for the arithmetic process can be selected, and the measurement data can be entered manually.

The target blood-sugar response curve can be created by various methods as mentioned above. By showing both the target blood-sugar response curve and the latest measurement data, data can be provided for use by patients and medical doctors.

In FIG. 13, the horizontal axis indicates time, setting a meal-start as the starting point. Such display can be shown by breakfast, lunch, and dinner.

The first display device 4 and the second display device 12 may show, other than the measurement date and the latest measurement-data, a target blood-sugar level after a predetermined time after meal (one hour and 52 minutes later in FIG. 12) calculated from the target blood-sugar response curve. The calculation method may be the same with the case in the predicted blood-sugar level after two hours in FIG. 11.

In FIG. 13, the blood-sugar level monitoring device A and the blood-sugar level processing device B are connected via the first interface unit 8 such as a memory card throttle and the second interface unit 14: the blood-sugar level monitoring device A by itself achieves functions similar to that of conventional blood-sugar meter, and may be provided with functions for calculating a predicted measurement value of two hours later, and for counting down till the measurement time, for example.

Additionally, since displaying the measurement data by the period of 24 hours reduces viewability, the second display device 12 may be provided with a function of scrolling in the horizontal axis (arrow) direction so that the measurement data of specific 2 to 6 hours can be displayed.

The blood-sugar level management system according to the present invention may be provided with, in addition to the management of the blood-sugar level as described in the above, functions to provide the patient with various data with relative to the measurement-data from external data and the like, such as blood-sugar measurement data (calorie vs. blood-sugar level) by regular meal, blood-sugar measurement data (calorie vs. blood-sugar level) by commercially available diabetic meal, a target blood-sugar level set by a medical doctor for a patient, accumulation of the past measurement data of the patient, consumed calories, meal calories, the PFC ratio, meal menu, the heart rate appropriate for the exercise-approach, and other various data needed by diabetic patients. Further, it can be provided with functions of notifying a meal timing and an exercise timing, and functions of notifying approval or disapproval of meal and exercise.

Further, in the blood-sugar level management system of the present invention, a data processing for reducing the number of measuring the blood-sugar level in a day can be carried out by the data-arithmetic-processing unit 11 of the blood-sugar level processing device B. For example, a blood-sugar level measurement timing in a day can be set to, a first point (for example, before breakfast: BB), a second point (for example, after an elapse of two hours from a start of breakfast: AB), a third point (for example, before lunch: BL), a fourth point (for example, after an elapse of two hours from a start of lunch: AL), a fifth point (for example, before dinner: BD), a sixth point (for example, after an elapse of two hours from a start of dinner: AD) and a seventh point (for example, before sleep: BT). In the present invention, by the data processing to be mentioned later, the number of measurement time, seven times per day, can be reduced.

Although these seven points can be changed as appropriate, generally, as in the above, the application of the seven points that is recommended by diabetes treatment research is preferable from the reasons that since blood-sugar levels change greatly during daytime due to a meal intake and the like, the blood-sugar measurement before and after meal is important, and the blood-sugar measurement before sleep is important for checking in advance the low blood-sugar condition during sleeping.

First, a monthly average of the blood-sugar level for each of the first point, the second point, the third point, the fourth point, the fifth point, the sixth point, and the seventh point (hereinafter referred to as “individual average value”) is obtained. Although the greater the number of the measurement n in a month, the better, the number may be 4 to 13 in days. Additionally, the number of measurement time n for each of the first to seventh points in a month is preferably the same.

In the example of Table 1, in October 2003, n times (day) of the blood-sugar level measurement are carried out at each of the first to seven points, and individual average value is obtained. The individual average value for the first point (BB) in October 2003 is 104.2. Additionally, the individual average values for each of the second to the seventh points is 134.0, 93.2, 152.2, 85.4, 156.4, and 99.4, respectively. The individual average is obtained similarly for other months as well. In the example of Table 1, 9-months measurement was carried out. Although data of a long period are used for the measurement-result in the case where an exercise-approach after meal is used, for those with stable life conditions, such seven-points measurement-data of a long period of time are unnecessary to be used.

Then, an average value of the blood-sugar level (individual average) for the first point, the second point, the third point, the fourth point, the fifth point, the sixth point, and the seventh point (seven-points-average value) of the above month is obtained.

In the example of Table 1, when the average value of the individual average values of the first to the seventh points in October 2003 is obtained, the seven-points-average-value of 117.8 is obtained. The seven-points-average-value of other months is obtained similarly.

Further, a correlation coefficient is obtained from the seven-points-average-value and the above individual average value. The correlation coefficient can be obtained by a method of Pearson's correlation coefficient (product-moment correlation coefficient).

In the example of Table 1, the correlation coefficient for the first point (BB) in nine months is 0.6121 by the Pearson's correlation coefficient calculation. Similarly, the correlation coefficient for other points are obtained. In the example of Table 1, the correlation coefficient of the sixth point (AD) is the maximum, and the correlation coefficient of the third point (BL) is the minimum.

The number of time for measuring the blood-sugar levels in a day is reduced by referring to the correlation coefficient obtained as in the above. For example, the points are preferably applied in the order from the largest correlation coefficient, but in accordance with lifestyles, such as in view of preventing skipping the measurement, the second candidate can be selected.

Additionally, for example, the points are preferably selected from the points that can be controlled by an ordinary businessman with a certain degree of effort. In the example of Table 1, considering that meal contents and amount can be managed, the second point (AB), the fourth point (AL), and the sixth point (AD) can be selected. Additionally considering that exercise can be done with some efforts, the first point (BB), the second point (AB), the sixth point (AD), and the seventh point (BT) can be selected. In view of management indicator in general blood-sugar level measurement, the first point (BB) is preferably utilized.

TABLE 1

| | | | | | | | | | | Correlation |
|---|---|---|---|---|---|---|---|---|---|---|
| | 2003/10 | 2003/11 | 2003/12 | 2004/1 | 2004/2 | 2004/3 | 2004/4 | 2004/5 | 2004/6 | Coefficient |
| First Point | 104.2 | 106.6 | 113.0 | 110.4 | 109.8 | 99.4 | 107.8 | 106.9 | 97.3 | 0.6121 |
| Before Breakfast (BB) | | | | | | | | | | |
| Second Point | 134.0 | 137.4 | 118.3 | 123.1 | 102.4 | 105.0 | 130.2 | 107.0 | 100.8 | 0.8112 |
| Morning 2H (AB) | | | | | | | | | | |
| Third Point | 93.2 | 94.0 | 97.3 | 88.4 | 92.4 | 83.7 | 90.6 | 93.7 | 93.7 | 0.3296 |
| Before Lunch (BL) | | | | | | | | | | |
| Forth Point | 152.2 | 119.3 | 128.8 | 127.0 | 146.0 | 117.7 | 123.1 | 129.8 | 126.0 | 0.5084 |
| Lunch 2H (AL) | | | | | | | | | | |
| Fifth Point | 85.4 | 91.1 | 79.8 | 77.0 | 72.1 | 71.5 | 78.4 | 77.8 | 77.8 | 0.5989 |
| Before Dinner (BD) | | | | | | | | | | |
| Sixth Point | 156.4 | 133.4 | 131.3 | 126.1 | 113.7 | 97.0 | 110.0 | 94.0 | 68.0 | 0.9681 |
| Dinner 2H (AD) | | | | | | | | | | |
| Seventh Point | 99.4 | 94.9 | 95.7 | 110.4 | 106.0 | 92.7 | 105.3 | 87.0 | 83.0 | 0.6173 |
| Before Sleep (BT) | | | | | | | | | | |
| Seven-Points-Average | 117.8 | 111.0 | 109.1 | 108.9 | 106.1 | 95.3 | 106.5 | 99.5 | 92.4 | 1.0000 |

Further, in the present invention, the number of measurement can be further reduced by processing data of the first point (BB), the second point (AB), the fourth point (AL), the sixth point (AD), and the seventh point (BT) selected from the example of Table 1.

To be specific, the average value of at least two individual average values from the first point (BB), the second Point (AB), the fourth point (AL), the sixth point (AD), and the seventh point (BT) (two-points-average-value, three-Points-average-value four-points-average-value, or five-points-average-value) is obtained.

Then, a correlation coefficient of each of these average values and the above seven-points-average-value is obtained by a method of the Pearson's correlation coefficient.

In the above example, as shown in Tables 2 and 3, there are 26 combinations for the first point (BB), the second point (AB), the fourth point (AL), the sixth point (AD), and the seventh point (BT). It is apparent that of these combinations, a correlation coefficient for a combination (BB, AB, AL, and AD), a combination (AB, AL, and AD), and a combination (BB, and AD) is high. In Tables 2 and 3, the combinations are listed in descending order, from the highest correlation coefficient on top.

TABLE 2

| | | | | | | | | | | Correlation |
|---|---|---|---|---|---|---|---|---|---|---|
| | 2003/10 | 2003/11 | 2003/12 | 2004/1 | 2004/2 | 2004/3 | 2004/4 | 2004/5 | 2004/6 | Coefficient |
| Four-points- | 136.7 | 124.2 | 122.8 | 121.6 | 118.0 | 104.8 | 117.8 | 109.4 | 98.0 | 0.9953 |
| aver. val. | | | | | | | | | | |
| BB, AB, AL, AD | | | | | | | | | | |
| Five-points- | 129.2 | 118.3 | 117.4 | 119.4 | 115.6 | 102.4 | 115.3 | 104.9 | 95.0 | 0.9902 |
| aver. val. | | | | | | | | | | |
| BB, AB, AL, AD, BT | | | | | | | | | | |
| Four-points- | 135.5 | 121.3 | 118.5 | 121.6 | 117.0 | 103.1 | 117.2 | 104.5 | 94.5 | 0.9875 |
| aver. val. | | | | | | | | | | |
| AB, AL, AD, BT | | | | | | | | | | |
| Three-points- | 147.5 | 130.1 | 126.1 | 125.4 | 120.7 | 106.6 | 121.1 | 110.3 | 98.3 | 0.9866 |
| aver. val. | | | | | | | | | | |
| AB, AL, AD | | | | | | | | | | |
| Three-points- | 130.1 | 121.1 | 120.0 | 120.2 | 119.4 | 107.3 | 120.4 | 114.6 | 108.0 | 0.9773 |
| aver. val. | | | | | | | | | | |
| BB, AB, AL | | | | | | | | | | |
| Three-points- | 131.5 | 125.8 | 120.9 | 119.9 | 108.6 | 100.5 | 116.0 | 102.6 | 88.7 | 0.9737 |
| aver. val. | | | | | | | | | | |
| BB, AB, AD | | | | | | | | | | |
| Two-points- | 130.3 | 120.0 | 122.2 | 118.2 | 111.8 | 98.2 | 108.9 | 100.5 | 82.7 | 0.9706 |
| aver. val. | | | | | | | | | | |
| BB, AD | | | | | | | | | | |
| Four-points- | 123.5 | 118.1 | 114.6 | 117.5 | 108.0 | 98.5 | 113.3 | 98.7 | 87.3 | 0.9706 |
| aver. val. | | | | | | | | | | |
| BB, AB, AD, BT | | | | | | | | | | |
| Three-points- | 129.9 | 121.9 | 115.1 | 119.9 | 107.4 | 98.2 | 115.2 | 96.0 | 83.9 | 0.9679 |
| aver. val. | | | | | | | | | | |
| AB, AD, BT | | | | | | | | | | |
| Two-points- | 145.2 | 135.4 | 124.8 | 124.6 | 108.0 | 101.0 | 120.1 | 100.5 | 84.4 | 0.9636 |
| aver. val. | | | | | | | | | | |
| AB, AD | | | | | | | | | | |
| Two-points- | 127.9 | 114.1 | 113.5 | 118.2 | 109.9 | 94.9 | 107.7 | 90.5 | 75.5 | 0.9599 |
| aver. val. | | | | | | | | | | |
| AD, BT | | | | | | | | | | |
| Four-points- | 122.5 | 114.5 | 113.9 | 117.7 | 116.0 | 103.7 | 116.6 | 107.7 | 101.8 | 0.9557 |
| aver. val. | | | | | | | | | | |
| BB, AB, AL, BT | | | | | | | | | | |
| Three-points- | 137.6 | 119.8 | 124.4 | 121.2 | 123.2 | 104.7 | 113.6 | 110.2 | 97.1 | 0.9554 |
| aver. val. | | | | | | | | | | |
| BB, AL, AD | | | | | | | | | | |

TABLE 3

| | | | | | | | | | | Correlation |
|---|---|---|---|---|---|---|---|---|---|---|
| | 2003/10 | 2003/11 | 2003/12 | 2004/1 | 2004/2 | 2004/3 | 2004/4 | 2004/5 | 2004/6 | Coefficient |
| Three-points- | 120.0 | 111.6 | 113.3 | 115.6 | 109.8 | 96.4 | 107.7 | 96.0 | 82.8 | 0.9549 |
| aver. val. | | | | | | | | | | |
| BB, AD, BT | | | | | | | | | | |
| Four-points- | 128.1 | 113.5 | 117.2 | 118.5 | 118.9 | 101.7 | 111.6 | 104.4 | 93.6 | 0.9514 |
| aver. val. | | | | | | | | | | |
| BB, AL, AD, BT | | | | | | | | | | |
| Three-points- | 136.0 | 115.9 | 118.6 | 121.2 | 121.9 | 102.5 | 112.8 | 103.6 | 92.3 | 0.9489 |
| aver. val. | | | | | | | | | | |
| AL, AD, BT | | | | | | | | | | |
| Three-points- | 128.5 | 117.2 | 114.2 | 120.2 | 118.1 | 105.1 | 119.5 | 107.9 | 103.3 | 0.9464 |
| aver. val. | | | | | | | | | | |
| AB, AL, BT | | | | | | | | | | |
| Two-points- | 154.3 | 126.4 | 130.0 | 126.5 | 129.9 | 107.3 | 116.6 | 111.9 | 97.0 | 0.9441 |
| aver. val. | | | | | | | | | | |
| AL, AD | | | | | | | | | | |
| Two-points- | 143.1 | 128.4 | 123.5 | 125.1 | 124.2 | 111.3 | 126.7 | 118.4 | 113.4 | 0.9399 |
| aver. val. | | | | | | | | | | |
| AB, AL | | | | | | | | | | |
| Three-points- | 112.5 | 112.9 | 109.0 | 114.6 | 106.1 | 99.0 | 114.4 | 100.3 | 93.7 | 0.8844 |
| aver. val. | | | | | | | | | | |
| BB, AB, BT | | | | | | | | | | |
| Two-points- | 119.1 | 122.0 | 115.6 | 116.7 | 106.1 | 102.2 | 119.0 | 107.0 | 99.1 | 0.8814 |
| aver. val. | | | | | | | | | | |
| BB, AB | | | | | | | | | | |
| Two-points- | 116.7 | 116.1 | 107.0 | 116.7 | 104.2 | 98.9 | 117.8 | 97.0 | 91.9 | 0.8653 |
| aver. val. | | | | | | | | | | |
| AB, BT | | | | | | | | | | |
| Three-points- | 118.6 | 106.9 | 112.5 | 115.9 | 120.6 | 103.3 | 112.1 | 107.9 | 102.1 | 0.7459 |
| aver. val. | | | | | | | | | | |
| BB, AL, BT | | | | | | | | | | |
| Two-points- | 125.8 | 107.1 | 112.2 | 118.7 | 126.0 | 105.2 | 114.2 | 108.4 | 104.5 | 0.6922 |
| aver. val. | | | | | | | | | | |
| AL, BT | | | | | | | | | | |
| Two-points- | 101.8 | 100.7 | 104.3 | 110.4 | 107.9 | 96.0 | 106.6 | 97.0 | 90.2 | 0.6756 |
| aver. val. | | | | | | | | | | |
| BB, BT | | | | | | | | | | |
| Two-points- | 128.2 | 112.9 | 120.9 | 118.7 | 127.9 | 108.5 | 115.5 | 118.4 | 111.7 | 0.6637 |
| aver. val. | | | | | | | | | | |
| BB, AL | | | | | | | | | | |

Based on such data processing, the number of measurement of the blood-sugar level in a day can be decreased by individuals, without reducing accuracy.

Additionally, in x-y coordinates, by obtaining a scatter diagram by plotting seven-points-average-value on x-axis, and by plotting four-points-average-value, three-points-average-value, or two-points-average-value on y-axis, the correlation of the seven-points-average-value with the four-points-average-value, the three-points-average-value, or the two-points-average-value can be confirmed visually. Then, by extrapolating a straight line illustrating the average (approximate straight line) in the scatter diagram, the degree of dispersion can be confirmed visually based on the shift from the straight line.

### INDUSTRIAL APPLICABILITY

A blood-sugar level measuring system in accordance with the present invention is a portable blood-sugar level monitoring device and can exhibit conventional blood-sugar level measuring functions, and enables management of effective data with the blood-sugar level processing device. Thus, management of meal intake timing and intake amount, management of blood-sugar level measurement timing, management of changes in blood-sugar level, and further a prediction of a future blood-sugar level, can be carried out precisely and easily for diabetic patients who are conducting exercise-approach or meal-approach in their daily lives away from medical institutes. Therefore, with an increase in diabetic patients, the device can be used suitably in diabetes treatment by medical doctors and medical institutes.

1. A blood-sugar level management system comprising:
a blood-sugar level monitoring device including
a blood-sugar monitoring unit for measuring a blood-sugar level of an examinee,
a measurement-data accumulation unit for accumulating measured blood-sugar level as measurement-data,
a measurement-control data-pre-processing unit for pre-processing said measurement-data,
a first display device for displaying the pre-processing result,
a first display-device control-unit for controlling said first display device,
a time-management unit for managing measurement timing of said blood-sugar level,
a control switch for controlling said monitoring unit, said measurement-data accumulation unit, said measurement-data pre-processing unit, said first display device, said first display-device control-unit, and said time-management unit, and
a first interface unit; and a blood-sugar level processing device including
a data-arithmetic-processing unit for arithmetically processing the measurement-data from said measurement-control data-pre-processing unit or external data,
a second display device for displaying the result of the arithmetic-processing,
a second display-device control-unit for controlling the second display device, and
a second interface unit;
wherein said blood-sugar monitoring device and said blood-sugar level processing device are connectible via said first interface unit and said second interface unit, and transmit to and receive from each other at least one of said measurement-data, the result of the pre-processing, and the result of the arithmetic-processing.

2. The blood-sugar level measuring system in accordance with claim 1, wherein said data-arithmetic-processing unit has a means for creating a graph in which the measured blood-sugar level is plotted against time based on said measurement-data, and said second display device has a means for displaying said graph.

3. The blood-sugar level measuring system in accordance with claim 1 or 2, wherein said data-arithmetic-processing unit is provided with a means for creating a graph in which said measured blood-sugar level is plotted against elapsed time from immediately after a start of a breakfast, lunch, or dinner intake based on said measurement-data; and said second display device is provided with a means for displaying said graph.

4. The blood-sugar level measuring system in accordance with claim 2 or 3, wherein said data-arithmetic-Processing unit is provided with a period-changing means for changing a period of said time by said control switch.

5. The blood-sugar level measuring system in accordance with any of claims 1 to 4, wherein said measurement-data accumulation unit is provided with a means for changing the accumulation period of said measurement-data.

6. The blood-sugar level measuring system in accordance with any of claims 1 to 5, wherein said external data is a target blood-sugar level set by a medical doctor.

7. The blood-sugar level measuring system in accordance with claim 6, wherein said second display device is provided with a means for plotting said target blood-sugar level on said graph in cooperation with said data-arithmetic-processing unit.

8. The blood-sugar level measuring system in accordance with any of claims 1 to 7, wherein said data-arithmetic-processing unit is provided with a means for creating a smoothed blood-sugar spline curve in which said measured blood-sugar level is plotted against time based on said measurement-data; and said second display device is provided with a means for displaying said smoothed blood-sugar spline curare on said graph.

9. The blood-sugar level measuring system in accordance with any of claims 1 to 8, wherein said data-arithmetic-processing unit is provided with a means for creating a target blood-sugar response curve showing a target blood-sugar level against time by assigning said measurement-data in a higher order approximation function; and said second display device is provided with a means for displaying said target blood-sugar response curve.

10. The blood-sugar level measuring system in accordance with claim 9, wherein the measurement-data to be assigned comprises three measured blood-sugar levels including at least a blood-sugar level at a meal-start and a blood-sugar level after an elapse of two hours from the meal-start.

11. The blood-sugar level measuring system in accordance with claim 9, wherein said measurement-data to be assigned comprises a blood-sugar level at a meal-start, a peak blood-sugar level, a time period from the meal-start to the point when the peak blood-sugar level is obtained, and a blood-sugar level after an elapse of two hours from the meal-start.

12. The blood-sugar level measuring system in accordance with claim 9 or 10, wherein said measurement-data to be assigned comprises a blood-sugar level at a meal-start, a blood-sugar level after an elapse of one hour from the meal-start, and a blood-sugar level after an elapse of two hours from the meal-start.

13. The blood-sugar level measuring system in accordance with any of claims 1 to 12, wherein said data-arithmetic-processing unit is provided with a means for creating a target blood-sugar response curve showing target blood-sugar levels against time by assigning the measurement-data accumulated in a certain period in the past in a higher order approximation function; and said second display device is provided with a means for displaying said target blood-sugar response curve.

14. The blood-sugar level measuring system in accordance with any of claims 1 to 13, wherein said data-arithmetic-processing unit is provided with a means for creating a target blood-sugar response curve showing target blood-sugar levels against time by assigning said external data in a higher order approximation function; and said second display device is provided with a means for displaying said target blood-sugar response curve.

15. The blood-sugar level measuring system in accordance with any of claims 1 to 14, wherein said data-arithmetic-processing unit is provided with a means for calculating a predicted blood-sugar level for after an elapse of a certain time based on the measurement-data accumulated in a certain period in the past or a latest measurement-data; and said second display device is provided with a means for displaying said predicted blood-sugar level.

16. The blood-sugar level measuring system in any of claims 1 to 15, wherein said control switch includes a timing input switch for inputting said measurement timing.

17. The blood-sugar level measuring system in accordance with claim 16, wherein said first display device is provided with a means for displaying said measurement timing or a time till said measurement timing in cooperation with said time-management unit.

18. The blood-sugar level measuring system in accordance with any of claims 1 to 17, wherein said first display device and/or said second display device is provided with a means for displaying a latest of said measured blood-sugar level by blinking or as a predetermined mark.

19. The blood-sugar level measuring system in accordance with any of claims 1 to 18, wherein said first display device and/or said second display device is provided with a means for scrolling the displayed contents in a horizontal direction or a vertical direction.

20. The blood-sugar level measuring system in accordance with any of claims 1 to 19, wherein said first display device and/or said second-display device is provided with a means for rotating the displayed contents upside-down.
